(19) United States
(12) Patent Application Publication
(10) Pub. No.: US 2022/0362213 A1
(43) Pub. Date: Nov. 17, 2022

(54) METHODS FOR TREATING POST ENDOSCOPIC RETROGRADE CHOLANGIOPANCREATOGRAPHY PANCREATITIS

(71) Applicants: Edward E. Purich, Brookeville, MD (US); Edward D. Purich, Silver Spring, MD (US)

(72) Inventors: Edward E. Purich, Brookeville, MD (US); Edward D. Purich, Silver Spring, MD (US)

(21) Appl. No.: 17/739,321

(22) Filed: May 9, 2022

Related U.S. Application Data
(60) Provisional application No. 63/189,376, filed on May 17, 2021.

(51) Int. Cl. A61K 31/405; A61K 31/196; A61K 9/00; A61P 1/18

(52) U.S. Cl. CPC A61K 31/405; A61K 31/196; A61K 9/006; A61P 1/18

(57) ABSTRACT

This invention relates generally to methods of treating post-endoscopic retrograde cholangiopancreatography pancreatitis. The method comprises sublingually or buccally administering to a patient deemed to be at risk of developing post endoscopic retrograde cholangiopancreatography pancreatitis prior to undergoing endoscopic retrograde cholangiopancreatography (ERCP), a therapeutically effective amount of a pharmaceutical composition comprising indomethacin or diclofenac or a combination thereof and a pharmaceutically acceptable carrier.

### CROSS-REFERENCE TO RELATED APPLICATIONS

This application claims priority to U.S. Provisional Patent Application Ser. No. 63/189,376 filed May 17, 2021, which is incorporated by reference in its entirety herein.

### BACKGROUND OF THE INVENTION

### 1. Field of the Invention

The invention relates generally to methods for treating post endoscopic retrograde cholangiopancreatography (ERCP) pancreatitis in patients. In this treatment, the method comprises administering sublingual or buccal doses of a therapeutically effective amount of a pharmaceutical composition comprising indomethacin or diclofenac or a combination thereof and a pharmaceutically acceptable carrier.

### 2. Brief Description of the Related Art

Endoscopic retrograde cholangiopancreatography (ERCP) is a commonly used diagnostic and therapeutic procedure that involves the injection of contrast and the performance of various therapeutic procedures (e.g., stone extraction, stent replacement, and the like). Over 700,000 such procedures are done annually in the U.S. Gastroenterologists perform this procedure for several reasons, including the evaluation of abdominal pain, the diagnosis of gallstones, the evaluation and diagnosis of diseases of the liver and pancreas, and to remove gallstones.

As with any medical procedure, there are risks associated with ERCP. Risks include punctures to the esophagus, bile duct or pancreatic duct, bleeding, and infection. The most common complication that can occur after ERCP is pancreatitis, commonly known as post-ERCP pancreatitis (Baillie J, Endoscopy 26:185-203, 1994; Sherman S, et al., Pancreas 6:350-367, 1991). Post-ERCP pancreatitis can cause a variety of conditions, including substantial morbidity, rare mortality, and increased costs. Its incidence ranges from 1-12% (mean approximately 7%), depending on the patient and the institution (Bilboa M K et al., Gastroenterology 70:314-320, 1976; Cotton P B, Gut 13:1014-25, 1972; Ruppin H et al., Endoscopy 6:94-8, 1974; Nebel O T et al., Gastrointest Endosc 22:34-6, 1975; LaFerla G et al., Pancreas 1:60-63, 1986; Rozler M H J et al., Radiology 157:595-8, 1985; Hamilton I et al., Clin. Radiol. 34:543-6, 1983; Brandes J W et al., Endoscopy 13:27-30, 1981; Nordback I et al., Ann. Chir. Gynaecol. 77:15-20, 1988). The incidence is higher in patients undergoing cholangiography with pancreatography than for those undergoing cholangiography alone, but is significant in both groups (Sherman et al., (1991); LaFerla G, et al., Pancreas 1:60-63 (1986); Skude G et al., Gut 17:127-32 (1976)). The severity of pancreatitis varies from mild, requiring minimal analgesia and outpatient management, to severe leading to the need for hospitalization, surgery, and in rare instances causing death.

A number of factors may play a role in the pathogenesis of post-ERCP pancreatitis. When associated with pancreatography, the pressure of the contrast injection alone can cause pancreatic injury and repeated injections are associated with a higher incidence of pancreatitis. The contrast agents themselves have been suggested to be potentially injurious; however, trials comparing ionic and non-ionic contrast have not shown a consistent difference in the incidence of post-ERCP pancreatitis.

Direct injection of contrast into the pancreatic duct is not the only factor involved in the genesis of post-ERCP pancreatitis; however, as those undergoing cholangiography alone are also subject to this complication. It is likely that irritation, inflammation or induced spasm of the sphincter of Oddi also plays a significant role. Of note, the incidence of post-ERCP pancreatitis increases with number of attempts at duct cannulation.

It is difficult to estimate the costs associated with the management of post-ERCP pancreatitis. Based on the assumption of a 1% risk of severe pancreatitis, a 3% risk of moderate pancreatitis and a 3% risk of mild pancreatitis, the costs can easily exceed 500 USD per patient. Combined with lost work and productivity, it is clear that an effective prophylactic regimen to prevent post-ERCP pancreatitis would be of great medical benefit.

Many medical interventions have been tried to prevent post-ERCP pancreatitis, including anticholinergics, antihistamines, corticosteroids, and antibiotics. Drugs that have been evaluated in controlled clinical trials include gabexate, somatostatin, octreotide, nifedipine, hydrocortisone, methylprednisolone, prednisone, interleukin-10, non-ionic contrast agents, glucagon, antibiotics, and calcitonin (Jowell P S et al. Gastroenterology 125(2):605, 2003; Renner I G et al., J. Clin. Invest. 72(3):1081-92, 1983; Niederau C et al., Gastroenterology 88(5 Pt 1): 1192-204, 1985; Renner I G et al., Dig. Dis. Sci. 31(3):305-13, 1986; Keim V et al., Hepatogastroenterology 32(2):91-6, 1985; Infantino A et al., Research in Experimental Medicine 190(2):89-93, 1990; Lankisch P G et al., Digestion 26(4):187-91, 1983; Manso M A et al., Peptides 10(2):255-60, 1989; Tymper F et al., Hepatogastroenterology 33(4):159-62, 1986; Kozarek R A et al., Gastrointest. Endosc. 51:AB138, 2000; Information from MD Consult Drug Information on Secretin. Mosby's Drug Consult (© 2003 Mosby, Inc.); Howard-McNatt M et al., Journal of Surgical Research 103; 96-99, 2002). Three agents—somatostatin, its octapeptide analog octreotide, and gabexate mesylate (a protease inhibitor) showed initial promise. Octreotide reduces hyperamylasemia but has not been shown to alter the clinical course of post-ERCP pancreatitis. A meta-analysis of 28 clinical trials showed that both prophylaxis with somatostatin and gabexate were effective in reducing the frequency of post-ERCP pancreatitis. However, in two large controlled trials in which pharmacologic prevention was provided to high-risk patients, gabexate, somatostatin and octreotide were each found to be ineffective in preventing post-ERCP pancreatitis. Additionally, a retrospective review of 4833 ERCP procedures suggested secretin might decrease the incidence of post-ERCP pancreatitis in patients without pancreas divisum (Mundorf J B et al., Am. J. Gastroenterology 90(9):1611, 1995; Mundorf J B et al., Am. J. Gastroenterology 90(9):1610, 1995).

Examples of treatments for post-ERCP pancreatitis in the patent literature include the following:

U.S. Pat. No. 6,143,306 assigned to Allergan Sales, Inc., discloses a non-radio therapy therapeutic method of treating disorders of the pancreas such as pancreatitis using a neurotoxin such as botulinum toxin.

U.S. Pat. No. 6,261,572 assigned to Allergan Sales, Inc., discloses a method for treating a pancreatic disorder by local administration of a therapeutic amount of a neurotoxin such as botulinum toxin, into or onto the body of the pancreas in order to treat symptoms of a pancreatic disorder.

U.S. Published Patent Application No. U.S. 2003/0132906 assigned to Schering-Plough Corporation, discloses the use of interleukin-10 (IL-10) for the prevention and treatment of pancreatitis. This patent discloses that IL-10 is administered to patients at risk of developing pancreatitis due to a procedure such as ERCP.

U.S. Pat. No. 5,094,837 assigned to Wayne State University discloses a method for using magnetic resonance imaging (MM) to image the pancreas by using secretin. An amount of secretin is placed in solution and administered to a patient for the purpose of pancreatic imaging. Administration of the secretin is done by IV infusion. The secretin employed in this method can be extracted from porcine or bovine sources or can be genetically recombined porcine, bovine or human secretin.

U.S. Pat. Nos. 6,020,310 and 6,498,143, both assigned to Repligen, disclose use of secretin to stimulate pancreatico-biliary fluid secretion in a patient exhibiting autism.

U.S. Pat. No. 6,197,746 assigned to Repligen Corporation discloses methods of using secretin for treating autism.

U.S. Pat. No. 6,365,593 assigned to Repligen Corporation discloses methods of diagnosing individuals for autistic disorders, comprising obtaining a sample of urine from the individuals; measuring a level of a methylxanthine in the urine sample; and comparing the level to a normal control or to a threshold level.

U.S. Pat. No. 6,534,063 to Joan Fallon discloses methods of utilizing the fecal chymotrypsin level of an individual as a measure of the success of secretin, other neuropeptides, and peptides or digestive enzyme administration to such individuals, and in particular, as a prognosticative of potential secretin, other neuropeptides, peptides, and digestive enzyme administration for persons having ADD, ADHD, Autism and other PDD related disorders.

U.S. Pat. No. 7,947,285 to Seymour Fein discloses methods for reducing the incidence of post-ERCP pancreatitis, consisting of the step of administering a therapeutically effective amount of a pharmaceutical composition comprising secretin and a pharmaceutically acceptable carrier.

The nonsteroidal anti-inflammatory compound indomethacin is known, and has been used for many years in various forms to treat pain. Similarly, diclofenac, sold under the brand name Voltaren among others, is a nonsteroidal anti-inflammatory drug (NSAID) used to treat pain and inflammatory diseases such as gout. It is taken by mouth, rectally in a suppository, used by injection, or applied to the skin.

While forms of indomethacin and diclofenac that are administered by mouth, such as capsules, oral solutions, and the like, are convenient for patients and healthcare providers, such administration methods necessarily subject the administered indomethacin to the upper gastrointestinal tract, including the liver, where the compound is deactivated and eliminated from the body before it can provide pain relief. Consequently, alternative administration methods for indomethacin have been investigated.

Recently, rectally administered nonsteroidal anti-inflammatory drugs, such as indomethacin (U.S. Pat. No. 3,849,549), have been used to reduce the incidence of pancreatitis after endoscopic retrograde cholangiopancreatography (ERCP) (Elmunzer B J, et al N. Engl. J. Med. (2012) 366:1414-1422). Subsequent studies continue to show that post ERCP Pancreatitis can be treated with doses of rectally administered indomethacin prior to or post ERCP procedure. See, for example Elmunzer B J, Hernandez I, Gellad W F, The Skyrocketing Cost of Rectal Indomethacin, Am. Med. Assn. Intern Med. 2020 May 1; 180(5):631-632 (2020); Feng Y, Navaneethan U, Zhu X, Varadarajulu S, Schwartz I, Hawes R, Hasan M, Yang A. Prophylactic rectal indomethacin may be ineffective for preventing post-endoscopic retrograde cholangiopancreatography pancreatitis in general patients: A meta-analysis, Dig. Endosc. 2017 May; 29(3):272-280; Patai Á, Solymosi N, Mohácsi L, Patai Á V, Indomethacin and diclofenac in the prevention of post-ERCP pancreatitis: a systematic review and meta-analysis of prospective controlled trials; Gastrointest Endosc. 2017 June; 85(6):1144-1156; Shi N, Deng L, Altaf K, Huang W, Xue P, Xia Q, Rectal indomethacin for the prevention of post-ERCP pancreatitis: A meta-analysis of randomized controlled trials; Turk J Gastroenterol. May 2015 6(3):236-40; Aziz M, Ghanim M, Sheikh T, Sharma S, Ghazaleh S, Fatima R, Khan Z, Lee-Smith W, Nawras A, Pancreatology April 2020; 20(3):356-361; Lindo Ricce M, Rodriguez López-Salazar T, Mendoza Jimenez-Ridruejo J, Moreno Monteagudo J A, Santander Vaquero C, Effectiveness of rectal indomethacin in the prevention of acute pancreatitis after endoscopic retrograde cholangiopancreatography in unselected patients, Rev Esp Enferm Dig. March 2020; 112(3):183-188; Wan J, Ren Y, Zhu Z, Xia L, Lu N, How to select patients and timing for rectal indomethacin to prevent post-ERCP pancreatitis: a systematic review and meta-analysis, BMC Gastroenterol. March 2017, 17(1):43; He X, Zheng W, Ding Y, Tang X, Si J, Sun L M, Rectal Indomethacin Is Protective against Pancreatitis after Endoscopic Retrograde Cholangiopancreatography: Systematic Review and Meta-Analysis, Gastroenterol Res Pract. May 9, 2018:9784841; Yaghoobi M, Alzahrani M A, McNabb-Baltar J, Martel M, Barkun A N, Rectal Indomethacin Prevents Moderate to Severe Post-ERCP Pancreatitis and Death and Should Be Used Before the Procedure: A Meta-Analysis of Aggregate Subgroup Data, J Can Assoc Gastroenterol. 2018 Mar. 27; 1(2):67-75.

NSAIDs including diclofenac or indomethacin have also been used for rectal administration, either pre-ERCP in 9 studies or post-ERCP in 5 studies, or during ERCP in 1 study (Hou et al. Sci. Rep. (2017) 7:46650). Diclofenac of approximately 100 mg were used in 15 studies and diclofenac 25 or 50 mg was used in 1 study (Otsuka et al., J. Gastroenterol. (2012) 47(8):912-917) for treatment of post-ERCP pancreatitis. However, while seemingly effective, rectal administration of indomethacin is inconvenient for patients and healthcare providers. In addition, the current cost of treating a patient suffering from post-ERCP pancreatitis is estimated at $15,000. Alternative and less expensive routes of administration of indomethacin for treating, for example, post-ERCP pancreatitis or other forms of pancreatitis or anti-inflammatory conditions, where the administered indomethacin is not deactivated by the upper GI tract is needed in the art. The present invention is believed to be an answer to that need.

### SUMMARY OF THE INVENTION

In one aspect, the present invention is directed to a method of treating post-endoscopic retrograde cholangiopancreatography pancreatitis, the method comprising the step of sublingually or buccally administering to a patient deemed to be at risk of developing post endoscopic retrograde cholangiopancreatography pancreatitis prior to undergoing endoscopic retrograde cholangiopancreatography (ERCP), a therapeutically effective amount of a pharmaceutical composition comprising indomethacin or diclofenac or a combination thereof and a pharmaceutically acceptable carrier.

In another aspect, the present invention is directed to a method for treating post-endoscopic retrograde cholangiopancreatography pancreatitis, consisting of the step of sublingually or buccally administering to a patient deemed to be at risk of developing post endoscopic retrograde cholangiopancreatography pancreatitis prior to undergoing endoscopic retrograde cholangiopancreatography (ERCP), a pharmaceutical composition comprising from about 1 to about 500 milligrams of indomethacin or diclofenac or a combination thereof and a pharmaceutically acceptable carrier, wherein said pharmaceutical composition is effective for reducing the incidence of post endoscopic retrograde cholangiopancreatography pancreatitis; and wherein said pharmaceutical composition is administered before any cannulation procedures.

These and other aspects will become apparent upon reading the following detailed description of the invention.

### DETAILED DESCRIPTION OF THE INVENTION

It has now been unexpectedly found that sublingual or buccal administration of indomethacin or diclofenac or a combination thereof is effective for treating or modulating the severity of post endoscopic retrograde cholangiopancreatography pancreatitis (post-ERCP pancreatitis). The treatment includes sublingually or buccally administering to a patient in need of such treatment a therapeutically effective amount of a pharmaceutical composition comprising indomethacin or diclofenac or a combination thereof and a pharmaceutically acceptable carrier prior, during, or immediately after ERCP is performed on that patient. Sublingual and buccal routes of administration of indomethacin or diclofenac or a combination thereof for treatment of conditions such as post-ERCP pancreatitis or other forms of pancreatitis or anti-inflammatory conditions, offers the advantage of fast and effective relief for these painful conditions, as well as the avoidance of unwanted side effects which can be a dangerous, or deadly affliction, during the ERCP procedure. Without wishing to be bound by any particular theory, it is believed that sublingual or buccal administration allows for indomethacin or diclofenac or a combination thereof to enter into the bloodstream directly and thereby bypass the gastrointestinal (GI) tract of the patient. In doing so, the indomethacin or diclofenac is not exposed to the body's digestive and filtering mechanisms such as found in the stomach and liver which would break down the indomethacin and reduce its effectiveness.

As indicated above, in one aspect, the present invention is directed to a method of treating post-endoscopic retrograde cholangiopancreatography pancreatitis, the method comprising the step of sublingually or buccally administering to a patient deemed to be at risk of developing post endoscopic retrograde cholangiopancreatography pancreatitis prior to undergoing endoscopic retrograde cholangiopancreatography (ERCP), a therapeutically effective amount of a pharmaceutical composition comprising indomethacin or diclofenac or a combination thereof and a pharmaceutically acceptable carrier. Each of these components and steps is explained in more detail below.

Indomethacin is a known and potent synthetic nonsteroidal indole derivative with anti-inflammatory activity and chemopreventive properties. Indomethacin is used to relieve pain, swelling, and joint stiffness caused by arthritis, gout, bursitis, tendonitis, and various other conditions. Nonsteroidal anti-inflammatory drugs (NSAIDs) are potent inhibitors of phospholipase A2, cyclooxygenase, and neutrophil-endothelial interactions, that are believed to play an important role in the pathogenesis of acute pancreatitis (Elmunzer B J, et al N. Engl. J. Med. 2012; 366:1414-1422). Meta-analysis of preliminary studies evaluating the protective effects of single-dose rectal indomethacin or diclofenac in post-ERCP pancreatitis have suggests benefit.

Diclofenac, sold under the brand name Voltaren among others, is a nonsteroidal anti-inflammatory drug (NSAID) used to treat pain and inflammatory diseases such as gout. The primary mechanism responsible for its anti-inflammatory, antipyretic, and analgesic action is thought to be inhibition of prostaglandin synthesis by inhibition of the transiently expressed prostaglandin-endoperoxide synthase-2 (PGES-2) also known as cyclooxygenase-2 (COX-2).

Indomethacin or diclofenac may be used individually or in a suitable combination, and combined with a pharmaceutically acceptable carrier to form a pharmaceutical composition. Thus, the pharmaceutical compositions of this invention comprise indomethacin from any source outlined above (including pharmaceutically acceptable salts thereof) in combination with any pharmaceutically acceptable carrier, adjuvant or vehicle. Pharmaceutically acceptable carriers, adjuvants and vehicles that may be used in the pharmaceutical compositions of the present invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride (saline), zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The indomethacin or diclofenac compounds of the invention are preferably administered either individually or in combination in a sublingual or buccal form and in an amount that produces acceptable bioavailability. In one embodiment, the compounds are administered preferably in a neat form or in a form of conventional pharmaceutical preparations, for example, in conventional water soluble pharmaceutically acceptable excipients containing organic and/or inorganic inert carriers, such as water, gelatin, saccharides, lactose, starch, magnesium stearate, talc, plant oils, gums, alcohol, Vaseline, or the like. Examples of some suitable disintegrator excipients are, croscarmellose sodium, sodium starch glycolate, and crospovidone. The non-limiting examples of some suitable pH adjusting excipients are sodium bicarbonate (NaHCO₃), calcium carbonate (CaCO₃), Na₂CO₃ (an alkalizing agent), and poloxamer 407 (a polymer used in the formulation of a nano-emulsifying solid dispersion formulations). Amorphous solid distribution carrier matrix materials such as PVP (polyvinyl pyrrolidone), HPMC (hydroxypropyl methylcellulose), HPMCP (hydroxypropyl methylcellulose phthalate), chitosan, CMC (carboxymethylcellulose), sodium alginate and sodium starch glycolate. The pharmaceutical preparations can be in conventional solid forms, for example, tablets, films, sprays, dragées, suppositories, capsules, amorphous solid dispersions, or the like, or conventional liquid forms, such as suspensions, emulsions, or the like. If desired, they can be sterilized and/or contain conventional pharmaceutical adjuvants, such as preservatives, stabilizing agents, wetting agents, emulsifying agents, buffers, or salts used for the adjustment of osmotic pressure. The pharmaceutical preparations may also contain other therapeutically active materials.

The pharmaceutical preparation of the invention should include an amount of indomethacin effective for preventing acute pancreatitis. In one embodiment, indomethacin, diclofenac, a combination thereof, or a pharmaceutical preparation of it, would be administered as soon after diagnosis of acute pancreatitis as possible and given daily (in divided doses) for about 3 days to about 14 days. The effective dosage will depend on several factors, including body weight, body mass index, age, gender and disease severity. Suitable dosages may be, for example, in the range of about 1-500 milligrams, or about 25 to about 250 milligrams, or about 50 to about 150 milligrams per dose. In addition, multiple doses of indomethacin and/or diclofenac may be required to be administered each day over a period of sublingually or buccally, up to four times per day for 7 days or more.

### Examples

The invention is further described by the following Examples, but is not intended to be limited by the Examples. All parts and percentages are by weight and all temperatures are in degrees Celsius unless explicitly stated otherwise.

The compositions and methods of the present invention are intended to preemptively treat patients at high or average risk of post-ERCP pancreatitis. As such, the compositions of the invention are intended to be administered either before or immediately following pancreatic surgery.

In one embodiment, an appropriate sublingual or buccal composition containing appropriate amounts of indomethacin, diclofenac or a combination thereof (generally ranging from 1-500 mg) is made using conventional production techniques known to those of skill in the art. The sublingual or buccal composition containing indomethacin, diclofenac, or a combination thereof is preferably administered to a human patient through a sublingual or buccal route prior to or immediately following pancreatic surgery. Administration may follow the following regimen: 1-500 mg two times a day, or 25-250 mg four times a day, or 50-150 mg four times a day.

### Patient Evaluation

The change in biochemical parameters that represent local and systemic effects of acute pancreatitis will be monitored, including changes from baseline values of C− reactive protein, pancreatic enzymes, sedimentation rate, hematocrit, APACHE II score, and oxygen saturation. Continuous monitoring of vital signs per standard of care and adverse events will also be monitored. Abdominal pain, elevated levels of amylase and lipase, inflammation, and fever are cardinal symptoms of pancreatitis and will be monitored and treated based on standard practice guidelines. Vital signs will be recorded at baseline prior to administration of study medication (indomethacin) and continuously throughout the study period per standard of care. A successful outcome will be indicated by no evidence of post-ERCP pancreatitis, or a reduction in symptoms, such as abdominal pain, elevated levels of amylase and lipase, inflammation, and fever.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entireties.

What is claimed is:

1. A method of treating post-endoscopic retrograde cholangiopancreatography pancreatitis, the method comprising the step of sublingually or buccally administering to a patient deemed to be at risk of developing post endoscopic retrograde cholangiopancreatography pancreatitis prior to undergoing endoscopic retrograde cholangiopancreatography (ERCP), a therapeutically effective amount of a pharmaceutical composition comprising indomethacin or diclofenac or a combination thereof and a pharmaceutically acceptable carrier.

2. The method of claim 1, wherein the method comprises the step of sublingual administration.

3. The method of claim 1, wherein the method comprises the step of buccal administration.

4. The method of claim 1, wherein the amount of indomethacin or diclofenac or combination thereof administered to said patient in said pharmaceutical composition ranges from about 1 to about 500 milligrams.

5. The method of claim 1, wherein the amount of indomethacin or diclofenac or combination thereof administered to said patient in said pharmaceutical composition ranges from about 25 to about 250 milligrams.

6. The method of claim 1, wherein the amount of indomethacin or diclofenac or combination thereof administered to said patient in said pharmaceutical composition ranges from about 50 to about 150 milligrams.

7. The method of claim 1, wherein said pharmaceutically acceptable carrier is selected from the group consisting of ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride (saline), zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat, and combinations thereof.

8. The method of claim 1, wherein said patient is a human.

9. A method for treating post-endoscopic retrograde cholangiopancreatography pancreatitis, consisting of the step of sublingually or buccally administering to a patient deemed to be at risk of developing post endoscopic retrograde cholangiopancreatography pancreatitis prior to undergoing endoscopic retrograde cholangiopancreatography (ERCP), a pharmaceutical composition comprising from about 1 to about 500 milligrams of indomethacin or diclofenac or a combination thereof and a pharmaceutically acceptable carrier, wherein said pharmaceutical composition is effective for reducing the incidence of post endoscopic retrograde cholangiopancreatography pancreatitis; and wherein said pharmaceutical composition is administered before any cannulation procedures.

10. The method of claim 9, wherein the method comprises the step of sublingual administration.

11. The method of claim 9, wherein the method comprises the step of buccal administration.

12. The method of claim 9, wherein said pharmaceutically acceptable carrier is selected from the group consisting of ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride (saline), zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat, and combinations thereof.

13. The method of claim 9, wherein the amount of indomethacin or diclofenac or combination thereof administered to said patient in said pharmaceutical composition ranges from 25 to about 250 milligrams.

14. The method of claim 9, wherein the amount of s indomethacin or diclofenac or combination thereof administered to said patient in said pharmaceutical composition ranges from about 50 to about 150 milligrams.

15. The method of claim 9, wherein said patient is a human.
